(19) United States
(12) Patent Application Publication
(10) Pub. No.: US 2014/0107536 A1
(43) Pub. Date: Apr. 17, 2014

(54) ULTRASONIC IRRADIATION APPARATUS AND METHOD FOR IRRADIATING ULTRASONIC WAVE

(71) Applicant: OLYMPUS CORPORATION, Tokyo (JP)

(72) Inventor: Miyuki MURAKAMI, Hachioji-shi (JP)

(73) Assignee: OLYMPUS CORPORATION, Tokyo (JP)

(21) Appl. No.: 14/104,504

(22) Filed: Dec. 12, 2013

Related U.S. Application Data
(63) Continuation of application No. PCT/JP2012/062938, filed on May 21, 2012.

(30) Foreign Application Priority Data
Jun. 15, 2011 (JP) 2011-133063

(51) Int. Cl. A61N 7/00

(52) U.S. Cl. CPC A61N 7/00
USPC 601/2

(57) ABSTRACT

An ultrasonic irradiation apparatus includes an ultrasonic emission part, an ultrasonic reception part, a low frequency signal detector, and a condition change unit. The ultrasonic emission part emits a first ultrasonic wave including a first frequency component having a frequency of f toward a target portion based on an ultrasonic setting value where f is a positive real number. The ultrasonic reception part receives a second ultrasonic wave which travels in a direction from the target portion. The low frequency signal detector detects a low frequency signal included in the second ultrasonic wave, the lower frequency signal having a frequency not greater than f/r where r is a real number and is greater than 2. The condition change unit changes the ultrasonic setting value based on the low frequency signal.

### CROSS-REFERENCE TO RELATED APPLICATIONS

This application is a Continuation Application of PCT Application No. PCT/JP2012/062938, filed May 21, 2012 and based upon and claiming the benefit of priority from prior Japanese Patent Application No. 2011-133063, filed Jun. 15, 2011, the entire contents of all of which are incorporated herein by reference.

### BACKGROUND OF THE INVENTION

1. Field of the Invention

The present invention generally relates to an ultrasonic irradiation apparatus and a method for irradiating an ultrasonic wave.

2. Description of the Related Art

When a medium is irradiated with an ultrasonic wave, a high negative pressure is generated in the medium and causes a cavitation. Owing to effects of a shock wave and a microjet caused by occurrence of the cavitation, for example, biological tissue can be broken and heating coagulation can be achieved. In recent years, attention is paid to a technology of applying breakdown and heating coagulation of biological tissue caused by a cavitation to therapeutic treatments.

For example, Japanese Patent No. 2741907 discloses a technology relating to a therapeutic ultrasonic apparatus. This therapeutic ultrasonic apparatus comprises a unit which intermittently irradiates a desired portion inside a living body with a converging first ultrasonic wave having a frequency f from a surface of the living body. The therapeutic ultrasonic apparatus locally anticancer-activates a photo-sensitizing substance or a substance capable of generating a chelate, which is injected into a living body, by irradiation with a converging ultrasonic wave. Further, a sub-harmonic component (having a frequency of f/2) or a high harmonic wave component (having a frequency of 2nf, where n is a natural number) of the first ultrasonic wave is radiated by a cavitation. Therefore, this therapeutic ultrasonic apparatus comprises a position detection means which receives the sub-harmonic component or the high frequency component and detects a position irradiated with an ultrasonic wave thereof. For example, Japanese Patent No. 4095729 discloses a technology relating to an ultrasonic apparatus for the purpose of medical treatments as follows. An irradiation position of a focused ultrasonic wave for a therapeutic treatment is displayed first on a screen display unit by a marking means. A transducer is fixed near a portion to be treated and is driven by a drive means. Also disclosed is a technology relating to a therapeutic ultrasonic apparatus, which stops irradiation of a focused ultrasonic wave when the irradiation position of the converging ultrasonic wave is away from the position of a transducer during irradiation of the converging ultrasonic wave for a therapeutic treatment.

### BRIEF SUMMARY OF THE INVENTION

According to an aspect of the invention, there is provided an ultrasonic irradiation apparatus including: an ultrasonic emission part configured to emit a first ultrasonic wave including a first frequency component having a frequency of f where the f is a positive real number based on an ultrasonic setting value, the first ultrasonic wave being emitted toward a target portion; an ultrasonic reception part configured to receive a second ultrasonic wave travelling in a direction from the target portion; a low frequency signal detector configured to detect a low frequency signal included in the second ultrasonic wave, the low frequency signal having a frequency of f/r where r is a real number and is greater than 2; and a condition change unit configured to change the ultrasonic setting value based on the low frequency signal.

According to another aspect of the invention, there is provided a method for irradiating an ultrasonic wave by use of an ultrasonic irradiation apparatus comprising an ultrasonic emission part configured to emit an ultrasonic wave and an ultrasonic reception part configured to receive an ultrasonic wave, the method including: setting an ultrasonic setting value including a frequency setting value for setting a first frequency f to be included in a first ultrasonic wave which is emitted from the ultrasonic emission part toward a target portion where the f is a real number; causing the ultrasonic emission part to emit the first ultrasonic wave based on the ultrasonic setting value; causing the ultrasonic reception part to receive a second ultrasonic wave travelling in a direction from the target portion; extracting a low frequency signal included in the second ultrasonic wave, the low frequency signal having a frequency not greater than f/r where r is a real number and is greater than 2; obtaining a peak value of the low frequency signal and a peak frequency corresponding to the peak value; determining an occurrence status of a cavitation in an area between the ultrasonic emission part and the target portion, by comparing the peak value and the peak frequency with a predetermined threshold; and changing the ultrasonic setting value based on a result of the determination.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 shows an example configuration of an ultrasonic irradiation apparatus according to the first embodiment;

FIG. 2 is a flowchart which shows an example operation of the ultrasonic irradiation apparatus according to the first embodiment;

FIG. 3 is a drawing for explaining an example of a group of cavitation bubbles which are generated by ultrasonic irradiation;

FIG. 4 is a graph showing an example of a relationship among time, frequency, and intensity of an ultrasonic wave which is received by an ultrasonic reception part, according to the first embodiment;

FIG. 5A is a graph showing an example of a relationship among time, frequency, and intensity of an ultrasonic wave at a time point t0, which is received by the ultrasonic reception part, according to the first embodiment;

FIG. 5B is a graph showing an example of a relationship among time, frequency, and intensity of the ultrasonic wave at time points t1 and t2, which is received by the ultrasonic reception part, according to the first embodiment;

FIG. 5C is a graph showing an example of a relationship among time, frequency, and intensity of the ultrasonic wave received by the ultrasonic reception part at time points t3 and t4 according to the first embodiment;

FIG. 6 is a graph showing an example of time-dependent changes of intensity at a first peak according to the first embodiment;

FIG. 7 is a flowchart showing an example operation of an ultrasonic irradiation apparatus, according to a modification of the first embodiment;

FIG. 8 is a graph showing an example of time-dependent changes of frequency at a first peak;

FIG. 9 shows an example configuration of an ultrasonic irradiation apparatus in the vicinity of a drive signal setting unit, according to the second embodiment of the invention;

FIG. 10 is a graph showing an example of a relationship among time, frequency, and intensity of an ultrasonic wave which is received by an ultrasonic reception part, according to the second embodiment;

FIG. 11 is a graph showing an example of time-dependent changes of intensity at first and second peaks;

FIG. 12 is a flowchart showing an example operation of the ultrasonic irradiation apparatus according to the second embodiment;

FIG. 13 is a graph showing an example of time-dependent changes of frequencies at first and second peaks;

FIG. 14 shows an example configuration of an ultrasonic irradiation apparatus in the vicinity of a drive signal setting unit, according to the second embodiment; and

FIG. 15 shows an example configuration of an ultrasonic irradiation apparatus according to the third embodiment.

### DETAILED DESCRIPTION OF THE INVENTION

### First Embodiment

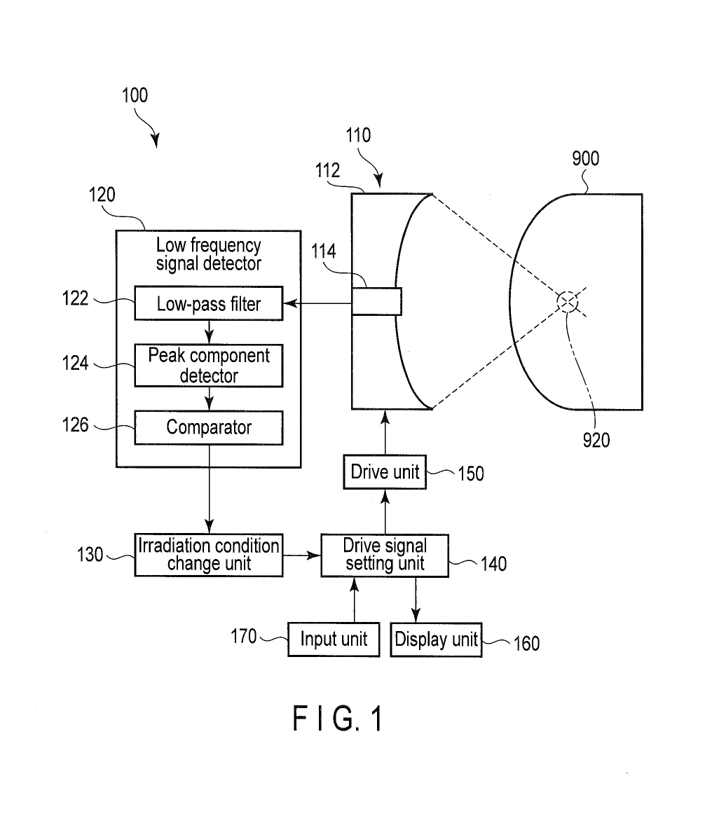

The first embodiment of the invention will now be described with reference to the drawings. An ultrasonic irradiation apparatus 100 irradiates a desired portion of an internal organ with a focused ultrasonic wave, for example, to heat and coagulate tissue of the desired portion. FIG. 1 shows a configuration of the ultrasonic irradiation apparatus 100. As shown in this figure, the ultrasonic irradiation apparatus 100 comprises an ultrasonic input/output unit 110, a low frequency signal detector 120, an irradiation condition change unit 130, a drive signal setting unit 140, a drive unit 150, a display unit 160, and an input unit 170.

The ultrasonic input/output unit 110 comprises an ultrasonic emission part 112 and an ultrasonic reception part 114. The ultrasonic emission part 112 is a piezoelectric element which has, for example, a concave surface shape. Electrodes not shown are formed respectively along concave and convex surfaces, so as to face each other, the piezoelectric element being located to the electrodes. The ultrasonic emission part 112 is driven by applying an alternating current voltage between the electrodes by the drive unit 150, and emits a focused ultrasonic wave (first ultrasonic wave). The ultrasonic emission part 112 is directed to, for example, an ultrasonic irradiation target 900. At this time, the focused ultrasonic wave converges on a focus 920 in the ultrasonic irradiation subject 900.

The ultrasonic reception part 114 includes, for example, a piezoelectric element which has wide band characteristics and functions as a hydrophone. That is, the ultrasonic reception part 114 receives sonic waves which are irradiated from the focus 920 and the vicinity thereof and enter the ultrasonic input/output unit 110. At this time, the received sonic waves include sonic waves of cavitation bubbles formed by the focused ultrasonic wave. The ultrasonic reception part 114 outputs signals corresponding to the received sonic waves, to the low frequency signal detector 120. The ultrasonic reception part 114 is provided, for example, in the center of an emission surface of the ultrasonic emission part 112. The position of the ultrasonic reception part 114 is not limited to the center of the ultrasonic emission part 112. The ultrasonic reception part 114 needs only to be configured to be capable of detecting sonic waves which travel from the ultrasonic irradiation target 900.

The low frequency signal detector 120 comprises a low-pass filter 122, a peak component detector 124, and a comparator 126. Received signals output from the ultrasonic reception part 114 are input to the low-pass filter 122. The low-pass filter 122 is set to allow a received signal having a frequency component not greater than a desired frequency to pass among the received signals input from the ultrasonic reception part 114. The desired frequency is set to, for example, f/4 where the frequency of an ultrasonic wave emitted from the ultrasonic emission part 112 is supposed to be f (f is a real number). The peak component detector 124 performs an FFT processing on a low frequency component of the received signal, namely, a low frequency signal which passes the low-pass filter 122. Thus, the peak component detector 124 calculates signal intensity for each frequency of low frequency signals or especially calculates a frequency at which a peak exists and intensity for the peak at each predetermined time point. The comparator 126 compares signal intensity for each frequency, which is calculated by the peak component detector 124, with a preset setting value, and outputs a result thereof to the irradiation condition change unit 130.

The irradiation condition change unit 130 outputs an instruction to stop irradiation of a focused ultrasonic wave or outputs an ultrasonic setting value (changed value) for the focused ultrasonic wave, to the drive signal setting unit 140, in accordance with input from the low frequency signal detector 120. Based on an instruction from a user input through the input unit 170, the drive signal setting unit 140 sets an ultrasonic setting value (initial value) for the focused ultrasonic wave, and creates a drive signal based on the frequency and intensity. Further, the drive signal setting unit 140 generates a drive signal, based on the ultrasonic setting value (changed value) input from the irradiation condition change unit 130. The drive signal setting unit 140 outputs the generated drive signal to the drive unit 150. When the drive signal setting unit 140 changes the irradiation condition of the focused ultrasonic wave on the basis of the signal input from the irradiation condition change unit 130, the drive signal setting unit 140 causes the display unit 160 to display content of a change to notify the user of the content. The content of a change may be notified in the form of a sound to the user. The drive unit 150 drives the ultrasonic emission part 112, based on the drive signal input from the drive signal setting unit 140. The ultrasonic setting value includes a type or value of an irradiation parameter, such as a frequency or intensity, or an operative method of a therapy or treatment. These values are irradiation conditions. The following description will be made with use of those terms.

The display unit 160 displays the irradiation conditions of the focused ultrasonic wave, etc., under control of the drive signal setting unit 140. The input unit 170 receives an instruction of a user and outputs the instruction to the drive signal setting unit 140. The user can know a status of the ultrasonic irradiation apparatus 100 and information concerning the focused ultrasonic wave, from the information displayed on the display unit 160. The user can input the information concerning start and end of irradiation of the focused ultrasonic wave and an ultrasonic setting value for the focused ultrasonic wave to the ultrasonic irradiation apparatus 100 through the input unit 170.

Thus, for example, the ultrasonic emission part 112 functions as an ultrasonic emission part which emits a first ultrasonic wave including a first frequency component having a frequency of f (where f is a positive real number), toward a target portion, based on the ultrasonic setting value. For example, the ultrasonic reception part 114 functions as an ultrasonic reception part which receives a second ultrasonic wave traveling in a direction from the target portion. For example, the low frequency signal detector 120 functions as a low frequency signal detector which detects a low frequency signal included in the second ultrasonic wave and having a frequency of f/r (where r is a real number greater than 2). For example, the irradiation condition change unit 130 functions as a condition change unit which changes a setting value on the basis of the low frequency signal. For example, the display unit 160 functions as a notification unit which notifies the user of a change to an ultrasonic setting value.

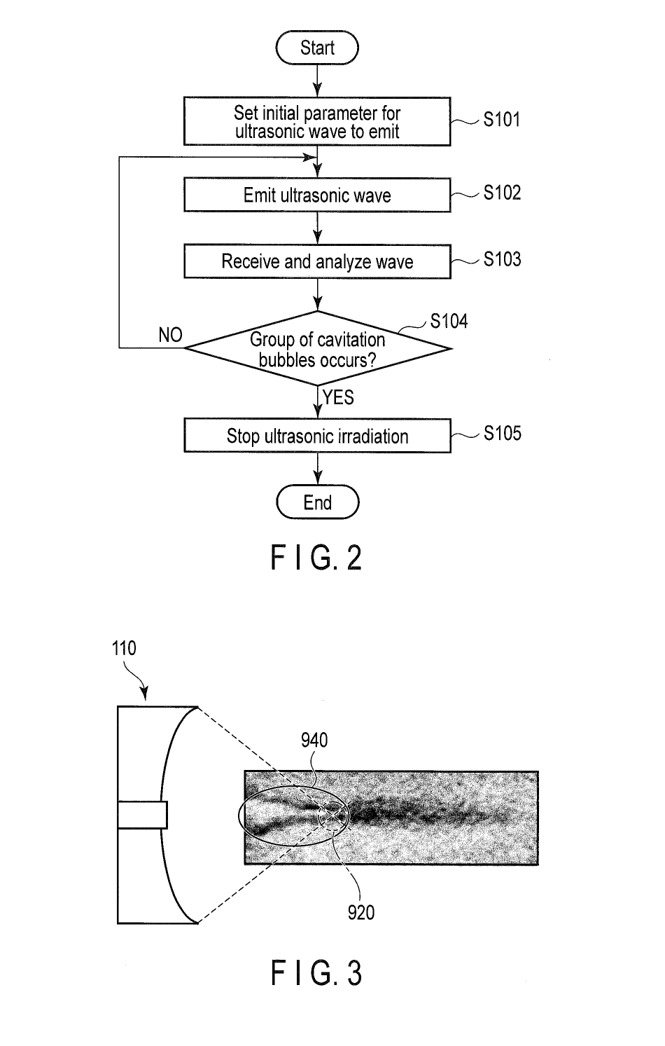

Operation of the ultrasonic irradiation apparatus 100 according to the present embodiment will be described with reference to a flowchart shown in FIG. 2. Firstly, the user directs the ultrasonic input/output unit 110 to face toward the ultrasonic irradiation target 900. A coupling material may be inserted between the ultrasonic irradiation target 900 and the ultrasonic input/output unit 110. This coupling material is for matching acoustic impedances of the ultrasonic irradiation target 900 and the ultrasonic input/output unit 110 with each other. Also, the ultrasonic irradiation target 900 may be applied with, for example, Sonazoid (registered trademark) as an ultrasonic contrast medium in advance.

In Step S101, the drive signal setting unit 140 obtains an instruction of the user from the input unit 170, and sets an ultrasonic setting value for the focused ultrasonic wave, based on the instruction. More details will now be described below. The user inputs, to the ultrasonic irradiation apparatus 100, the ultrasonic setting value for the focused ultrasonic wave which is irradiated on the ultrasonic irradiation target 900, by using the input unit 170. As described previously, the ultrasonic setting value expresses a type and/or a value of an irradiation parameter, and/or an operative method of a therapy or treatment. For example, the user may directly input (select) a type and an initial value of an irradiation parameter. Alternatively, the user may input an operative method of a therapy or treatment, and the drive signal setting unit 140 may set a type and an initial value of an irradiation parameter. The input unit 170 outputs an input ultrasonic setting value to the drive signal setting unit 140. The drive signal setting unit 140 sets a type and an initial value of an irradiation parameter for the focused ultrasonic wave, based on the ultrasonic setting value input from the input unit 170. The drive signal setting unit 140 generates a drive signal to be output to the drive unit 150, based on the set initial value. Types of irradiation parameters are not limited to the intensity and frequency of ultrasonic waves.

In Step S102, the drive signal setting unit 140 outputs a drive signal to the drive unit 150, and causes the ultrasonic emission part 112 to emit a focused ultrasonic wave from the ultrasonic emission part 112. More details will now be described below. The user inputs, to the ultrasonic irradiation apparatus 100 (drive signal setting unit 140), an instruction to start emission of a focused ultrasonic wave by using the input unit 170. The drive signal setting unit 140 to which the instruction to start emission of a focused ultrasonic wave is input outputs a drive signal to the drive unit 150. The drive unit 150 amplifies the drive signal input from the drive signal setting unit 140, and applies an alternating current signal based on the drive signal to the ultrasonic emission part 112. The ultrasonic emission part 112 is driven based on the alternating current signal applied from the drive unit 150. That is, the ultrasonic emission part 112 vibrates. As a result, a focused ultrasonic wave is emitted from the ultrasonic emission part 112 toward the ultrasonic irradiation target 900. The frequency of the focused ultrasonic wave is set to f (where f is a real number).

The focused ultrasonic wave converges on the focus 920. Irradiation of the focused ultrasonic wave causes cavitation at the focus 920. At the focus 920, for example, tissue coagulates by heating due to the cavitation. FIG. 3 shows an example of a status of a group of cavitation bubbles generated by irradiation of a focused ultrasonic wave. The image shown in FIG. 3 is an image obtained by an ultrasonic diagnostic apparatus immediately after irradiation of the focused ultrasonic wave is stopped after irradiation for a while. In FIG. 3, the focus 920 is a portion which is to be heated and coagulated by causing cavitation. When ultrasonic irradiation is continued for a long time, a group of more cavitation bubbles then occurs in an area 940 between the ultrasonic input/output unit 110 and the focus 920. In the image of FIG. 3, the group of cavitation bubbles is observed in a white color and spreads in a fan-like shape from the focus 920 toward the left side. The group of cavitation bubbles increases in quantity as the irradiation time of the focused ultrasonic wave elapses. The group of cavitation bubbles immediately disappears upon stoppage of irradiation of the focused ultrasonic wave, and transits into a status that bubbles cannot be observed by an ultrasonic diagnostic apparatus.

Cavitation bubbles each of which has such a size that cannot be observed in an image obtained by the ultrasonic diagnostic apparatus exhibits an effect of proceeding heating and coagulation of tissue at the focus 920. On the other hand, a group of cavitation bubbles is formed when cavitation bubbles each grow to such a size that can be observed in an image obtained by the ultrasonic diagnostic apparatus. In this status, the area 940 between the ultrasonic input/output unit 110 and the focus 920 is more heated and coagulated. That is, damage may be inflicted on tissue of a portion which is not to be subjected to a therapeutic treatment. Therefore, in order to perform an optimal therapy or treatment, the output intensity of the focused ultrasonic wave needs to be changed or emission of the focused ultrasonic wave needs to be stopped, depending on the status of cavitation bubbles. In the present embodiment, emission of the focused ultrasonic wave is stopped on the basis of information of a sonic wave received by the ultrasonic reception part 114.

In Step S103, the low frequency signal detector 120 analyzes a received sonic wave. More details will now be described below. The ultrasonic reception part 114 receives a sonic wave which travels in a direction from the focus 920. Especially, the ultrasonic reception part 114 receives sonic waves originated from a group of cavitation bubbles. The ultrasonic reception part 114 outputs received signals to the low frequency signal detector 120.

In the low frequency signal detector 120, the low-pass filter 122 extracts a received signal having a frequency not greater than a desired frequency (a low frequency signal), among received signals input from the ultrasonic reception part 114. The low-pass filter 122 outputs the extracted low frequency signal to the peak component detector 124. The peak component detector 124 performs FFT analysis on the low frequency signal which passes through the low-pass filter 122, and calculates a signal intensity for each frequency or, more specifically, a frequency at a peak and intensity thereof, at each predetermined time point. The peak component detector 124 outputs the frequency and intensity to a comparator 126.

In the present embodiment, as an example, a low frequency signal having a frequency not higher than a frequency of f/4 is extracted, and occurrence of a group of cavitation bubbles is determined by analyzing the low frequency signal. Therefore, the low-pass filter 122 is a low pass filter which allows a low frequency signal having a frequency not higher than f/4 to pass. The cutoff frequency of the low-pass filter 122 is not limited to this frequency but needs only to be lower than f/2. However, the cutoff frequency of the low-pass filter 122 is desirably f/5 or higher.

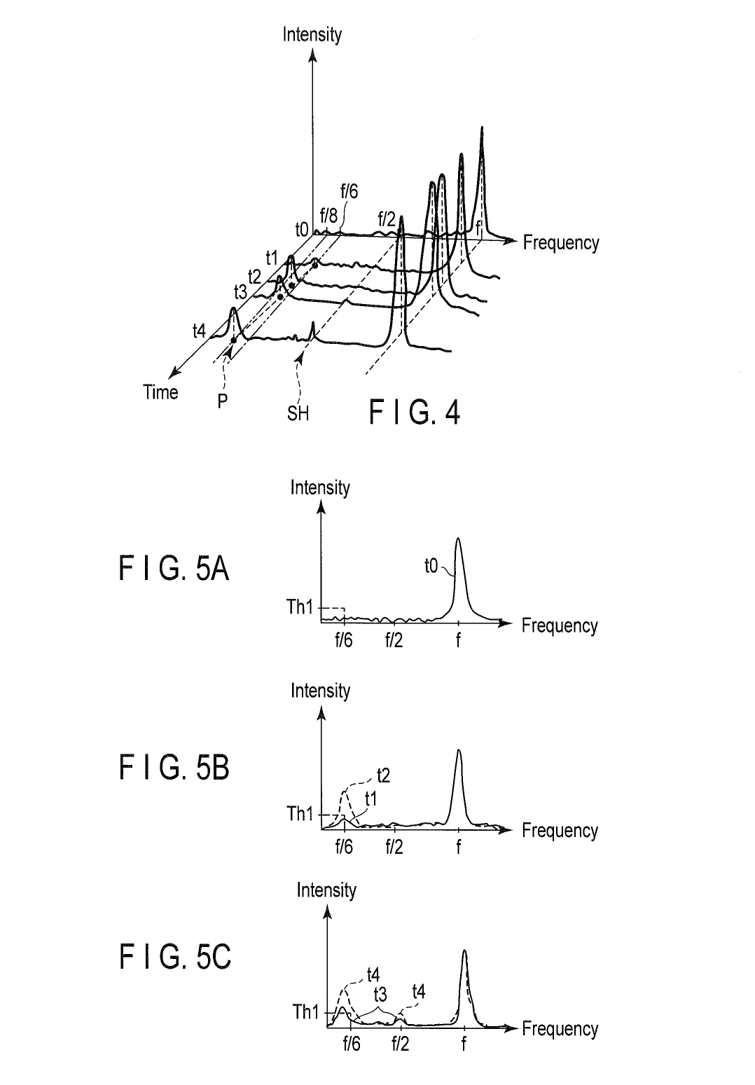

FIGS. 4, 5A, 5B, and 5C show examples of time-dependent changes of the frequency and intensity of a sonic wave, which is generated from the group of cavitation bubbles among sonic waves received by the ultrasonic reception part 114. FIG. 4 shows a relationship among elapsed time since start of irradiation, frequency, and intensity, in the form of a three-dimensional graph. FIGS. 5A, 5B, and 5C show a relationship between the frequency and the intensity in the form of a two-dimensional graph at each of irradiation time points. The frequency of the focused ultrasonic wave, i.e., the drive frequency is f. The irradiation time is configured to elapse through t1, t2, t3, and t4 from an irradiation start time point t0. FIG. 5A shows a relationship between frequency and intensity at the time point t0. FIG. 5B shows a relationship between frequency and intensity at the time points t1 and t2. FIG. 5C shows a relationship between frequency and intensity at the time points t3 and t4.

At the time of starting irradiation of a focused ultrasonic wave (time point t0), only the peak corresponding to the drive frequency of f is detected by the ultrasonic reception part 114. At the time point t1 when a certain time elapses from the start of irradiation, a first peak P is observed near the frequency of f/6 which is approximately ⅙ of the drive frequency, in addition to the component of the drive frequency of f. Occurrence of the first peak P originates from occurrence of a group of cavitation bubbles. Furthermore, at the time t2 when a much longer time elapses, the intensity at the first peak P is higher than at the time point t1. Hence, the group of cavitation bubbles is understood to be growing.

As time further elapses (to the time points t3 and t4), the frequency at the first peak P gradually decreases. For example, when ultrasonic irradiation is continued for several tens of seconds, the frequency at the first peak P decreases from f/6 nearly to f/8. Thus, the frequency at the first peak P decreases continuously as time elapses. Such a decrease in frequency at the first peak P is caused by great differences in size among bubbles of a group of cavitation bubbles. Therefore, a strong correlation exists between the frequency at the first peak P and growth of the group of cavitation bubbles. If the irradiation time of the focused ultrasonic wave increases, another peak is also observed near the frequency of f/2. The other peak of the frequency f/2 is equivalent to a sub-harmonic wave SH of the drive frequency f. The peak near the intensity of the frequency of f/2, namely, the intensity of the sub-harmonic wave SH is lower than the intensity at the first peak P.

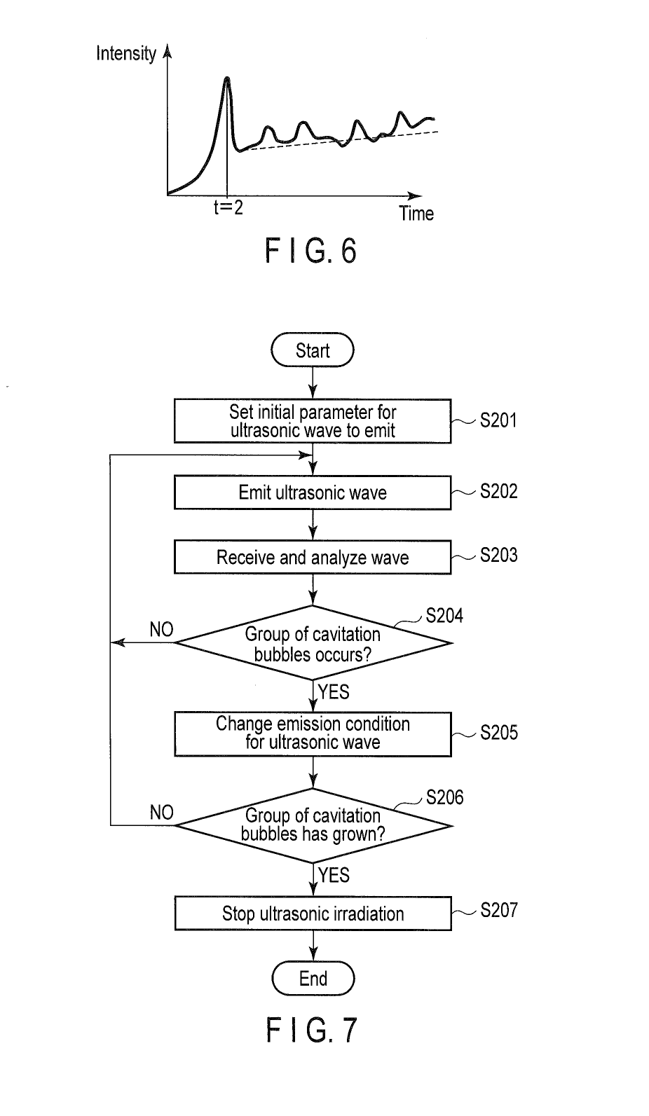

FIG. 6 shows time-dependent changes of the intensity at the first peak P. As shown in this figure, the intensity rises once at the time t2 when cavitation occurs, and the intensity then drops. While the intensity at the first peak P changes and goes up and down after the drop, a baseline of changes rises gradually, as indicated by a broken line shown in FIG. 6.

In Step S104, the comparator 126 of the low frequency signal detector 120 determines whether a group of cavitation bubbles has occurred or not based on an obtained low frequency signal. More details will now be described below. For example, as shown in FIGS. 5A, 5B, and 5C, a threshold value Th1 is set in the comparator 126. When the intensity at the first peak P of a low frequency signal detected by the peak component detector 124, i.e., the intensity at a peak near the frequency of f/6 increases to be higher than the threshold Th1, the comparator 126 determines that a group of cavitation bubbles occurs. The threshold value Th1 is set to a value which is sufficiently higher than a noise level. For example, the threshold Th1 may be twice greater than a noise level.

Although FIGS. 4, 5A, 5B, and 5C show the peak at the frequency of f which is the peak of the drive frequency and f/2 which is a sub-harmonic wave of the drive frequency of f, the low-pass filter 122 cuts off signals having, for example, frequencies not lower than f/4 through the processing by the low frequency signal detector 120. That is, a signal having the drive frequency of f and a signal having the frequency of f/2 which is sub-harmonic of the drive frequency can be prevented from being input to the peak component detector 124 by setting a cutoff frequency of the low-pass filter 122 to be smaller than f/2.

If any group of cavitation bubbles is not determined to be occurring by the determination in Step S104, processing returns to S102 where the ultrasonic irradiation apparatus 100 continues irradiation of a focused ultrasonic wave without changing irradiation conditions of the focused ultrasonic wave.

Otherwise, if a group of cavitation bubbles is determined to be occurring by the determination in Step S104, the ultrasonic irradiation apparatus 100 stops irradiation of the focused ultrasonic wave in Step S105. More details will now be described below. Information that a group of cavitation bubbles occurs is input to the irradiation condition change unit 130 from the comparator 126. The irradiation condition change unit 130 outputs, to the drive signal setting unit 140, an instruction to stop emission of the focused ultrasonic wave emitted from the ultrasonic emission part 112. The drive signal setting unit 140 stops outputting of a drive signal to the drive unit 150, based on the instruction from the irradiation condition change unit 130. As a result, the ultrasonic emission part 112 stops emission of the focused ultrasonic wave. At this time, the drive signal setting unit 140 causes the display unit 160 to indicate that emission of the focused ultrasonic wave is to be stopped. Thereafter, the ultrasonic irradiation apparatus 100 terminates the processing.

According to the present embodiment, the ultrasonic irradiation apparatus 100 can detect occurrence of a group of cavitation bubbles in the area 940 closer to the side of the ultrasonic input/output unit 110 than the focus 920 as the low frequency signal detector 120 analyzes sonic waves received by the ultrasonic reception part 114. If occurrence of a group of cavitation bubbles is detected, the ultrasonic irradiation apparatus 100 stops irradiation of the focused ultrasonic wave. By stopping irradiation of the focused ultrasonic wave, the area 940 closer to the side of the ultrasonic input/output unit 110 than the focus 920 is not irradiated with the focused ultrasonic wave. Therefore, tissue other than at a target position which is to be, for example, heated and coagulated can be prevented from being damaged.

### Modification of First Embodiment

A modification of the first embodiment will now be described below. Differences from the first embodiment will be described herein. The same parts as those of the first embodiment will be denoted with the same reference signs, respectively, and detailed descriptions thereof will be omitted. In the present modification, when occurrence of a group of cavitation bubbles is detected, an irradiation condition of a focused ultrasonic wave is changed. When growth of the group of cavitation bubbles is detected thereafter, irradiation of the focused ultrasonic wave is stopped.

FIG. 7 shows a flowchart which expresses an example of processings according to the present modification. Steps S201, S202, and S203 in the present modification are the same as Steps S101, S102, and S103 in the first embodiment described with reference to FIG. 2, respectively. That is, in Step S201, the drive signal setting unit 140 obtains user instruction information, for example, from the input unit 170. Further, a type of an irradiation parameter and an initial value thereof for a focused ultrasonic wave to be emitted from the ultrasonic irradiation apparatus 100 is set based on the user instruction information. In Step S202, the drive signal setting unit 140 outputs a drive signal to the drive unit 150, and causes the ultrasonic emission part 112 to emit a focused ultrasonic wave from the ultrasonic emission part 112. In Step S203, the ultrasonic reception part 114 receives a sonic wave, and the low frequency signal detector 120 analyzes the received sonic wave.

In Step S204, the comparator 126 of the low frequency signal detector 120 determines whether a group of cavitation bubbles occurs or not based on an obtained low frequency signal. More details will now be described below. For example, a threshold Th1 is set in the comparator 126 in advance, as in the first embodiment. When the intensity at the first peak of a low frequency signal detected by the peak component detector 124, i.e., the intensity at the first peak near the frequency of f/6 increases to be higher than the threshold Th1, the comparator 126 determines that a group of cavitation bubbles occurs.

If any group of cavitation bubbles is not determined to be occurring by the determination in Step S204, processing returns to S202. The ultrasonic irradiation apparatus 100 continues emission of a focused ultrasonic wave without changing irradiation conditions for the focused ultrasonic wave. Otherwise, if a group of cavitation bubbles is determined to be occurring in Step S204, the drive signal setting unit 140 changes an irradiation condition for the focused ultrasonic wave of the ultrasonic wave in Step S205. More details will now be described below.

The irradiation condition change unit 130 changes the irradiation conditions, based on a comparison result input from the low frequency signal detector 120. More specifically, a changed value of the drive signal is calculated with a type and a value of an irradiation parameter changed. The irradiation condition change unit 130 outputs the calculated changed value of the drive signal to the drive signal setting unit 140, and simultaneously outputs, to the drive signal setting unit 140, an instruction to change the drive signal on the basis of the changed value. For example, the irradiation condition change unit 130 outputs the changed value of the drive signal to the drive signal setting unit 140 so as to lower the intensity of the focused ultrasonic wave. The irradiation condition change unit 130 may determine a change value so as to set the intensity of the focused ultrasonic wave to preset intensity, or may determine a change value so as to set intensity based on a first peak detected by the peak component detector 124.

The drive signal setting unit 140 generates a drive signal, which is changed based on the change value input from the irradiation condition change unit 130. The drive signal setting unit 140 outputs the changed drive signal to the drive unit 150. The drive unit 150 drives the ultrasonic emission part 112, based on the changed signal. As a result, the ultrasonic emission part 112 emits, for example, a changed focused ultrasonic wave whose intensity has dropped. At this time, the drive signal setting unit 140 displays content of the change on the display unit 160 to report the content to the user.

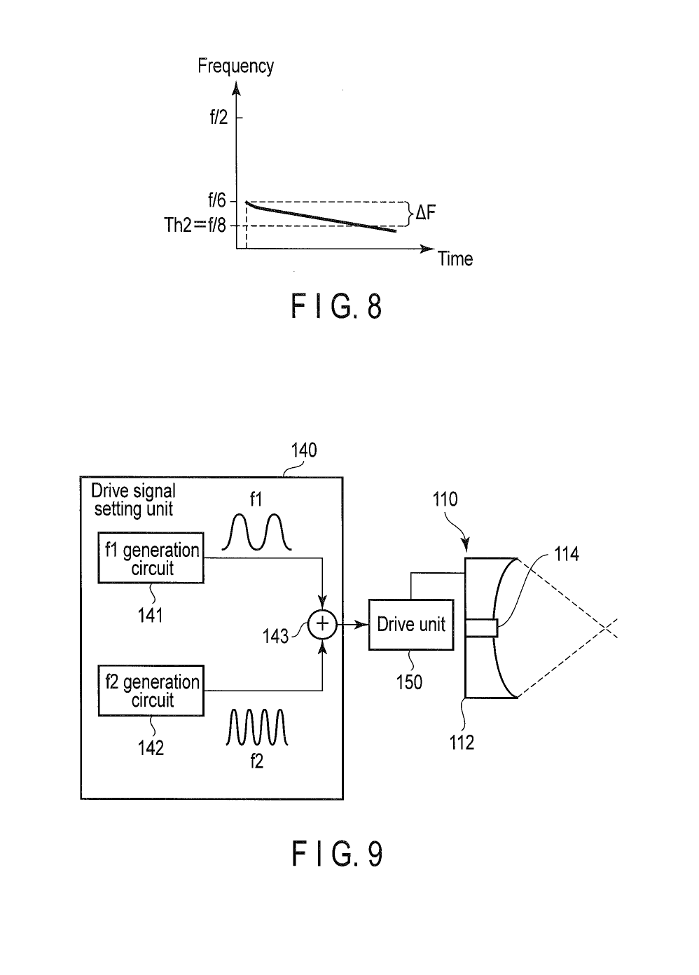

In Step S206, the comparator 126 of the low frequency signal detector 120 determines whether a group of cavitation bubbles has grown or not based on an obtained low frequency signal. More details will now be described below. FIG. 8 shows time-dependent changes of the frequency at the first peak. As described above, the first peak of a low frequency signal which originates from a group of cavitation bubbles is detected near the frequency of f/6 and then gradually drops as time elapses. In Step S206, the comparator 126 detects growth of a group of cavitation bubbles, based on the drop of the frequency at the first peak. For example, a threshold Th2 is set for the frequency at the first peak. When the frequency at the first peak decreases to be lower than the threshold Th2, the group of cavitation bubbles is determined to have grown. Here, the threshold Th2 can be set to a frequency of f/8, for example, as shown in FIG. 8.

If no group of cavitation bubbles is determined to have grown by the determination in Step S206, processing returns to Step S202. As a result, according to the illumination conditions changed in Step S205, the ultrasonic emission part 112 emits a focused ultrasonic wave in Step S202. If any group of cavitation bubbles is determined to have grown by the determination in Step S206, processing proceeds to Step S207. In Step S207, the irradiation condition change unit 130 instructs the drive signal setting unit 140 to stop irradiation of the focused ultrasonic wave, as in Step S105 in the first embodiment. As a result, the ultrasonic emission apparatus 100 stops emission of the focused ultrasonic wave. Then, a series of processings ends.

In some cases, occurrence of a group of cavitation bubbles near the frequency of f/6 does not immediately adversely influence tissue other than at the target position, e.g., tissue which is originally not to be heated or coagulated. In such cases, according to the present modification, irradiation of a focused ultrasonic wave can be continued for a while from occurrence of a group of cavitation bubbles, i.e., while irradiation of the focused ultrasonic wave is effective and does not adversely influence tissue other than at a target position. Thereafter, transition to a status in which a group of cavitation bubbles has grown and adversely influences tissue other than at the target position is detected by checking a change in frequency at the first peak detected by the low frequency signal detector 120, and is notified to the irradiation condition change unit 130. As a result, the irradiation condition change unit 130 can instruct the drive signal setting unit 140 to stop emission of the focused ultrasonic wave.

In the present modification, when the frequency at the first peak drops to be lower than the threshold Th2, a group of cavitation bubbles is determined to have grown. However, in Step S206, for example, when a frequency change ΔF at the first peak shown in FIG. 8 increases to be greater than a predetermined threshold Th3, a group of cavitation bubbles may be determined to have grown in Step S206.

In the determination of Step S206, emission of the focused ultrasonic wave may be stopped not based on the detection of a change in frequency of a low frequency signal but based on elapse of a predetermined time period from detection of occurrence of a group of cavitation bubbles. The aforementioned predetermined time period may be set to 1 second or so.

As indicated by a broken line in FIG. 6, the intensity at a first peak increases gradually. Hence, in the determination of Step S206, a time-average value of the intensity at the first peak may be obtained at predetermined time intervals. Emission of the focused ultrasonic wave may be stopped when the time average intensity increases to be not smaller than a predetermined threshold.

According to any of the methods as described above, irradiation of the focused ultrasonic wave can be stopped not immediately after occurrence of a group of cavitation bubbles but before a group of bubbles grows and adversely influences tissue other than at the target position. Alternatively, by employing any of a plurality of determination conditions as described above, the intensity of the focused ultrasonic wave may be decreased when a predetermined condition is satisfied. When another predetermined condition is satisfied, emission of the focused ultrasonic wave may be stopped. Such a combination of determination conditions as described is appropriately selectable depending on designs and service conditions of the ultrasonic irradiation apparatus 100.

### Second Embodiment

The second embodiment will now be described.

Differences from the first embodiment will be described herein. The same parts as those of the first embodiment will be denoted with the same reference signs, respectively, and detailed descriptions thereof will be omitted. In the present embodiment, a drive signal on which two different frequencies are superimposed is applied to an ultrasonic emission part 112. In the present embodiment, two drive frequencies are set to f1=4.64 MHz and f2=9.28 MHz, for example. A drive signal setting unit 140 generates signals having two types of frequencies, respectively, and thereafter synthesizes the signals with each other to generate a drive signal, which is applied to the ultrasonic emission part 112 through a drive unit 150.

FIG. 9 shows a configuration of an area including the drive signal setting unit 140 according to the present embodiment. As shown in this figure, the drive signal setting unit 140 comprises an f1 generation circuit 141, an f2 generation circuit 142, and an adder 143. The f1 generation circuit 141 generates and outputs a signal having a frequency of f1. The f2 generation circuit 142 generates and outputs a signal having a frequency of f2. The signal having the frequency of f1 output from the f1 generation circuit 141 and the signal having the frequency of f2 output from the f2 generation circuit 142 are input into the adder 143. The adder 143 superimposes the signal output from the f1 generation circuit 141 and the signal output from the f2 generation circuit 142 on each other. The drive signals superimposed by the adder 143 are output to the drive unit 150. In the same manner as in the first embodiment, the drive unit 150 drives the ultrasonic emission part 112, based on the drive signal input from the drive signal setting unit 140.

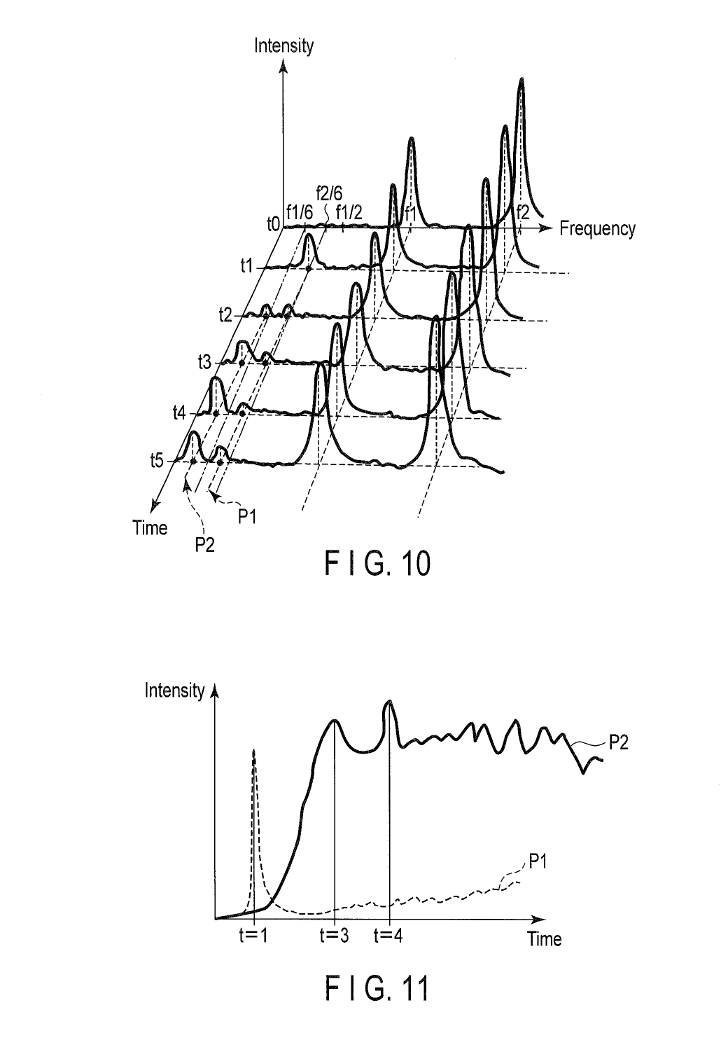

FIG. 10 shows an example of time-dependent changes of the frequency and intensity of a sonic wave, which is generated from a group of cavitation bubbles among sonic waves received by the ultrasonic reception part 114. FIG. 10 shows a relationship among elapsed time since start of irradiation, frequency, and intensity in the form of a three-dimensional graph. In the present embodiment, the frequencies of f1 and f2 are drive frequencies, and also are frequencies of sonic waves received by the ultrasonic reception part 114. At the time of starting irradiation of a focused ultrasonic wave (time point t0), only peaks corresponding to the drive frequencies of f1 and f2 are detected by the ultrasonic reception part 114. At a time point t1 when a certain time elapses from the start of ultrasonic irradiation, a first peak P1 is observed at a frequency of f2/6 which is approximately ⅙ of the drive frequency of f2, in addition to the peaks at the drive frequencies of f1 and f2. Occurrence of the first peak P1 originates from occurrence of a group of cavitation bubbles. The time point t1 is a timing equivalent to the time point t2 in the first embodiment (see FIG. 4).

At a time point t2 when time further elapses, the intensity at the first peak P1 is lower. At the time point t2, a second peak P2 is detected near the frequency of f1/6, in addition to the first peak P1. A sonic wave component at the second peak P2 originates from occurrence of a group of cavitation bubbles. As time further elapses through time points t3, t4, and t5, the intensity at the second peak P2 gradually rises while the frequency gradually drops. On the other hand, the frequency at the first peak P1 hardly changes but the intensity rises slightly.

FIG. 11 shows time-dependent changes of the intensities at the first peak P1 and second peak P2. As shown in this figure, at the time point t1 when cavitation occurs, a high intensity is observed at the first peak P1 and then drops. Thereafter, the intensity at the first peak P1 increases gently. On the other hand, the intensity at the second peak P2 gradually increases after the time point t1 and is saturated.

The first peak P1 and second peak P2 relate not only to the size of the group of cavitation bubbles but also to behaviors thereof. That is, not only occurrence of a group of cavitation bubbles but also a status thereof can be determined by monitoring two different low frequency signals at the first peak P1 and the second peak P2. For example, where a therapy to locally break tissue in an ultrasonic irradiation target 900 is supposed, there is a need to try to generate a group of cavitation bubbles. Whether a status of an occurring group of bubbles is suitable for performing the therapy or not can be determined by monitoring the two different low frequency components. That is, a group of cavitation bubbles can be effectively used safely.

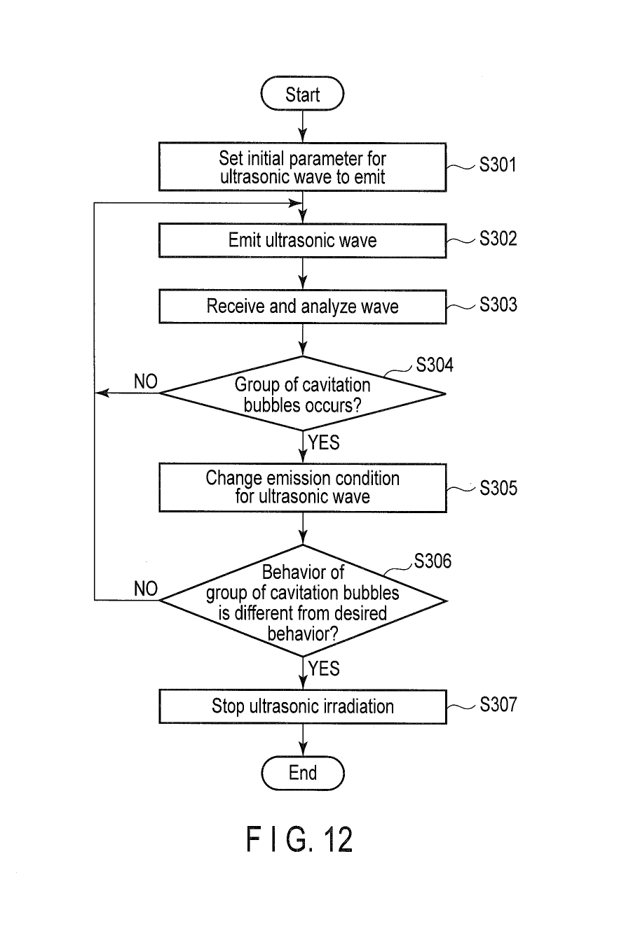

FIG. 12 shows a flowchart of an example of processings according to the present embodiment. Steps S301, S302, S303, S304, and S305 in the present embodiment are the same as Steps S201, S202, S203, S204, and S205 in the modification of the first embodiment described with reference to FIG. 7, respectively. In Step S301, the drive signal setting unit 140 obtains an instruction from a user through, for example, an input unit 170, and sets a type of an irradiation parameter of a focused ultrasonic wave, such as frequency or intensity, and an initial value thereof, based on the instruction. In Step S302, the drive signal setting unit 140 outputs a drive signal to a drive unit 150 so as to emit a focused ultrasonic wave. In Step S303, the ultrasonic reception part 114 receives sonic waves, and the low frequency signal detector 120 analyzes the received sonic waves.

In Step S304, the comparator 126 of the low frequency signal detector 120 determines whether a group of cavitation bubbles occurs or not based on an obtained low frequency signal. More details will now be described below. For example, a threshold Th1 is set in the comparator 126 in advance, as in the first embodiment. When the intensity at the first peak P1 of a low frequency signal detected by the peak component detector 124, i.e., the intensity at the first peak P1 near the frequency of f2/6 increases to be higher than the threshold Th1, the comparator 126 determines that a group of cavitation bubbles occurs.

If a group of cavitation bubbles is not determined to be occurring by the determination in Step S304, processing returns to S302 in which the ultrasonic irradiation apparatus 100 continues irradiation of a focused ultrasonic wave. Otherwise, if a group of cavitation bubbles is determined to be occurring in Step S304, the drive signal setting unit 140 changes a condition of the focused ultrasonic wave, based on settings of the irradiation condition change unit 130, in Step S305.

In Step S306, the comparator 126 of the low frequency signal detector 120 determines whether a behavior of a group of cavitation bubbles is different from a desired behavior or not based on an obtained low frequency signal. When, for example, the second peak P2 near the frequency of f1/6 is detected, a behavior of a group of cavitation bubbles is determined to be different from a desired behavior. Alternatively, when the frequency at the second peak P2 decreased to be lower than the threshold Th2 which is set near the frequency of f1/8, a behavior of a group of cavitation bubbles is determined to be different from a desired behavior.

If a behavior of a group of cavitation bubbles is not determined to be different from the desired behavior by the determination in Step S306, processing returns to Step S302. As a result, in accordance with an irradiation condition changed in Step S305, the ultrasonic emission part 112 emits a focused ultrasonic wave in Step S302. Otherwise, if a behavior of a group of cavitation bubbles is determined to be differed from the desired behavior by the determination in Step S306, the ultrasonic irradiation 130 instructs the drive signal setting unit 140 to stop irradiation of the focused ultrasonic wave. As a result, the ultrasonic emission apparatus 100 stops emission of the focused ultrasonic wave. Then, a series of processings ends.

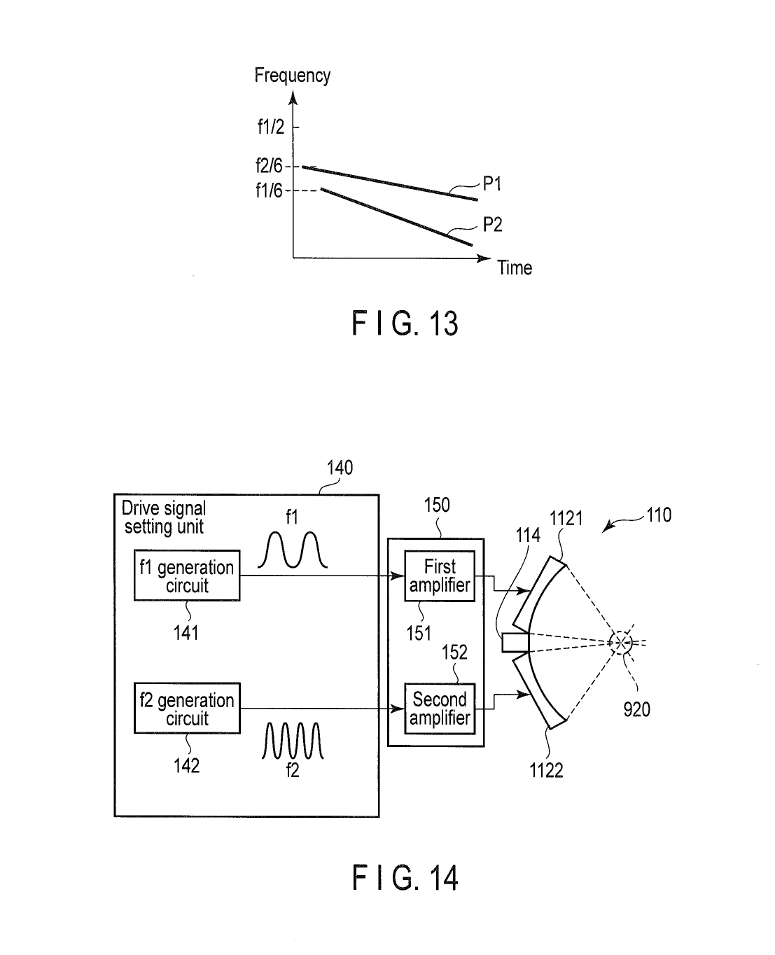

FIG. 13 shows time-dependent changes of frequencies, for example, at the first peak P1 and second peak P2. As described above, the first peak P1 which originates from a group of cavitation bubbles is detected near the frequency of f2/6. In Step S304, whether there is occurrence of a group of cavitation bubbles or not is determined based on whether the first peak has been detected or not. Thereafter, the frequency at the first peak gradually decreases as time elapses. After a while from detection of the first peak P1, the second peak P2 lower than the first peak P1 is detected. The frequency at the second peak P2 also gradually decreases as time elapses. A drop rate of the frequency at the second peak P2 is greater than that at the first peak P1. In Step S306, the comparator 126 determines whether a behavior of a group of cavitation bubbles is different from a desired behavior or not based on whether the second peak P2 has been detected or not. Alternatively, whether a behavior of a group of cavitation bubbles is different from a desired behavior or not is determined based on whether the frequency at the second peak P2 has decreased to be lower than the threshold Th2 or not.

In the determination in Step S306, when the frequency at the second peak P2 has decreased by a predetermined value or more, a behavior of the group of cavitation bubbles may be determined to be different from a desired behavior. Alternatively, when a predetermined time period elapses from detection of the first peak P1 or the second peak P2, a behavior of a group of cavitation bubbles may be determined to be different from the desired behavior. Still alternatively, when the intensity at the first peak P1 and/or the second peak P2 increases to be a specified value or greater, a behavior of a group of cavitation bubbles may be determined to be different from the desired behavior. If a safer irradiation condition is to be set in use of the ultrasonic irradiation apparatus 100, emission of a focused ultrasonic wave may be configured to be stopped when the first peak P1 is detected beyond the threshold Th1.

The present embodiment has been described with reference to a case that a relationship of f2=2*f1 is satisfied by a combination of two different drive frequencies f1 and f2. However, arbitrary combinations of the drive frequencies f1 and f2 may be available. Herein, a pass band of a low-pass filter 122 may be set to be not higher than the frequency of a sub-harmonic wave of the higher one of the two drive frequencies. That is, a cutoff frequency of the low-pass filter 122 may be set to a frequency of f2/2 or lower.

The same effects as in the first embodiment or the modification thereof are obtained also in the present embodiment. Further, not only occurrence of a group of cavitation bubbles but also a status thereof can be determined by monitoring two different low frequency signals at the first peak P1 and the second peak P2. Accordingly, an occurring group of cavitation bubbles can be effectively used safely.

### Modification of Second Embodiment

A modification of the second embodiment will now be described below. Differences from the second embodiment will be described herein. The same parts as those of the first embodiment will be denoted with the same reference signs, respectively, and detailed descriptions thereof will be omitted. In the second embodiment, a focused ultrasonic wave on which drive frequencies f1 and f2 are superimposed is emitted from one single ultrasonic emission part 112. In contrast, in the present modification, the ultrasonic input/output unit 110 comprises a first ultrasonic emission part 1121 which emits a focused ultrasonic wave having a frequency of f1, and a second ultrasonic emission part 1122 which emits a focused ultrasonic wave having a frequency of f2.

FIG. 14 shows a configuration of an area including a drive signal setting unit 140 in an ultrasonic irradiation apparatus 100 according to the present modification. As shown in this figure, the drive signal setting unit 140 comprises an fl generation circuit 141, and an f2 generation circuit 142. A drive unit 150 comprises a first amplifier 151 and a second amplifier 152. An ultrasonic input/output unit 110 comprises the first ultrasonic emission part 1121 and the ultrasonic reception ultrasonic wave emission part 1122.

A drive signal having the drive frequency of f1 generated by the f1 generation circuit 141 is input to the first amplifier 151. The amplified drive signal having the drive frequency of f1 input to the first amplifier 151 is further input to the first ultrasonic emission part 1121. As a result, the first ultrasonic emission part 1121 emits a focused ultrasonic wave having the drive frequency of f1. A drive signal having the drive frequency of f2 generated by the f2 generation circuit 142 is input to the second amplifier 152. The amplified drive signal having the drive frequency of f2 input to the f2 amplifier 152 is further input to the second ultrasonic emission part 1122. As a result, the second ultrasonic emission part 1122 emits a focused ultrasonic wave having the drive frequency of f2.

The first ultrasonic emission part 1121 and the second ultrasonic emission part 1122 are arranged in a manner that the focused ultrasonic wave emitted from the first ultrasonic emission part 1121 and the focused ultrasonic wave emitted from the second ultrasonic emission part 1122 cross each other at the focus 920. Therefore, at the focus 920, the focused ultrasonic wave having the frequency of f1 and the focused ultrasonic wave of the frequency f2 are superimposed on each other. As a result, at the focus 920 where the signals having the frequencies of f1 and f2 are superimposed, the same effects as in the second embodiment are obtained.

The configuration according to the present modification operates in the same manner and obtains the same effects as in the second embodiment.

### Third Embodiment

The third embodiment will now be described. Differences from the first embodiment will be described herein. The same parts as those of the first embodiment will be denoted with the same reference signs, respectively, and detailed descriptions thereof will be omitted. In the present embodiment, an ultrasonic input/output unit 110 is arranged at a distal end of a flexible endoscope, and the flexible endoscope further comprises a mechanism for applying Sonazoid (registered trademark) as an ultrasonic contrast medium to an ultrasonic irradiation target area (equivalent to the ultrasonic irradiation target 900 in FIG. 1). Sonazoid (registered trademark) is known to have a resonance frequency near 4.2 to 4.8 MHz although there is a distribution of particle sizes to some extent.

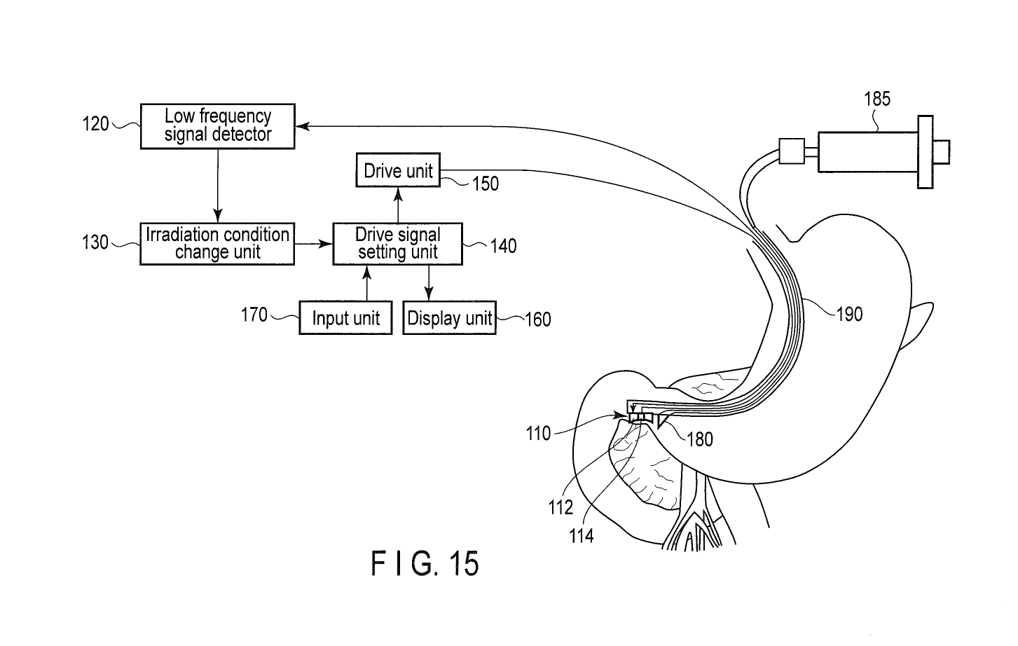

FIG. 15 shows a configuration of an ultrasonic treatment apparatus according to the present embodiment. As shown in this figure, the ultrasonic treatment apparatus according to the present embodiment comprises an injection unit. An ultrasonic input/output unit 110 is arranged at the distal end of a flexible endoscope 190 which is inserted orally. The ultrasonic input/output unit 110 comprises an ultrasonic emission part 112 and an ultrasonic reception part 114. A drive unit 150 connected to the ultrasonic emission part 112 and a low frequency signal detector 120 connected to the ultrasonic reception part 114 are arranged at a proximal end side of the endoscope 190. The ultrasonic emission part 112 and the drive unit 150 are connected to each other by a wiring into which the endoscope 190 is inserted. The ultrasonic reception part 114 and the low frequency signal detector 120 are connected to each other by a wiring into which the endoscope 190 is inserted. As in the first embodiment, an irradiation condition change unit 130 is connected to the low frequency signal detector 120. The drive signal setting unit 140 is connected to the irradiation condition change unit 130. The drive unit 150 is connected to a drive signal setting unit 140. In addition, a display unit 160 and an input unit 170 are connected to the drive signal setting unit 140.

Further, a puncturing unit 180 is arranged near the ultrasonic input/output unit 110 at the distal end of the endoscope 190. A pressure unit 185 arranged at the proximal end side of the endoscope 190 is connected to the puncturing unit 180. The puncturing unit 180 can feed an ultrasonic contrast medium supplied through the pressure unit 185, to the vicinity of the focus 920 of the focused ultrasonic wave which the ultrasonic emission part 112 emits. Thus, the puncturing unit 180 and the pressure unit 185 function as an injection unit which injects, into a target portion, a micro substance over which reflects or scatters an ultrasonic wave. The other features of the configuration are the same as those of the first embodiment. At this time, a frequency f of an ultrasonic wave to irradiate is desirably set to a resonance frequency of Sonazoid (registered trademark) or a frequency of a higher order than the resonance frequency. In addition, an application position of an ultrasonic contrast medium by the puncturing unit 180 and a focal position of a focused ultrasonic wave are desirably arranged in the rear side of the center of the therapeutic target area in relation to a sound source. By setting such a positional relationship as described, a high therapeutic effect can be obtained while reducing a shielding effect owing to the contrast media.

According to the present embodiment, for example, a pancreas and a gallbladder can be irradiated with a focused ultrasonic wave, for example, over an alimentary canal. Further, the ultrasonic contrast medium can be applied to only the vicinity of the focus 920 of the focused ultrasonic wave by the puncturing unit 180. Therefore, an effect of enhancing heating is expected from ultrasonic irradiation to a local position. At this time, the same effects as in the first or second embodiment can be obtained by driving the ultrasonic irradiation apparatus in the same manner as in the first and second embodiment.

As in the second embodiment, the ultrasonic emission part 112 may be driven at drive frequencies f1 and f2. The endoscope 190 is not limited to a flexible endoscope but a rigid endoscope may be used. In addition, a medical fluid to be injected is not limited to an ultrasonic contrast medium but may be a substance which reflects an ultrasonic wave, such as nano-bubbles or micro particulates of gold. When a substance which reflects an ultrasonic wave is applied, a portion applied with the substance easily causes cavitation and allows a reflected ultrasonic wave to be used more effectively. The ultrasonic contrast medium can also be applied by an intravenous injection.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic irradiation apparatus comprising:
an ultrasonic emission part configured to emit a first ultrasonic wave including a first frequency component having a frequency of f where the f is a positive real number based on an ultrasonic setting value, the first ultrasonic wave being emitted toward a target portion;
an ultrasonic reception part configured to receive a second ultrasonic wave travelling in a direction from the target portion;
a low frequency signal detector configured to detect a low frequency signal included in the second ultrasonic wave, the low frequency signal having a frequency of f/r where r is a real number and is greater than 2; and
a condition change unit configured to change the ultrasonic setting value based on the low frequency signal.

2. The ultrasonic irradiation apparatus of claim 1, wherein the ultrasonic setting value is changed so as to stop emission of the first ultrasonic wave.

3. The ultrasonic irradiation apparatus of claim 1, wherein the ultrasonic setting value is changed so as to lower intensity of the first ultrasonic wave.

4. The ultrasonic irradiation apparatus of claim 1, wherein the condition change unit changes the ultrasonic setting value when the low frequency signal detector detects the low frequency signal having intensity not smaller than a predetermined threshold.

5. The ultrasonic irradiation apparatus of claim 1, wherein the condition change unit changes the ultrasonic setting value when a predetermined time period elapses since the low frequency signal detector detects the low frequency signal having intensity not smaller than a predetermined threshold.

6. The ultrasonic irradiation apparatus of claim 1, wherein the condition change unit changes the ultrasonic setting value when the low frequency signal detector detects the low frequency signal having intensity not smaller than a predetermined threshold and detects a decrease of the frequency of the low frequency signal by a predetermined value.

7. The ultrasonic irradiation apparatus of claim 1, wherein the condition change unit changes the ultrasonic setting value when the low frequency signal detector detects the low frequency signal having intensity not smaller than a predetermined threshold and detects the frequency of the low frequency signal to be not greater than a predetermined value.

8. The ultrasonic irradiation apparatus of claim 1, wherein the condition change unit changes the ultrasonic setting value when the low frequency signal detector detects the low frequency signal having intensity not smaller than a predetermined threshold and detects a change of the intensity of the low frequency signal by a predetermined value.

9. The ultrasonic irradiation apparatus of claim 1, wherein the r is 4.

10. The ultrasonic irradiation apparatus of claim 1, wherein the first ultrasonic wave includes the first frequency component whose frequency is the f and a second frequency component whose frequency is f′ where the f′ is a positive real number and is smaller than f.

11. The ultrasonic irradiation apparatus of claim 1, wherein
the first ultrasonic wave includes the first frequency component whose frequency is the f and a second frequency component whose frequency is f′ where the f′ is a positive real number and is smaller than f,
the low frequency signal includes a first low frequency signal and a second low frequency signal, and
the condition change unit changes the ultrasonic setting value when the low frequency signal detector detects the first low frequency signal having a frequency lower than f/r and having intensity not smaller than a predetermined threshold and the second low frequency signal having a frequency lower than f′/r and having intensity not smaller than a predetermined threshold.

12. The ultrasonic irradiation apparatus of claim 1, wherein
the first ultrasonic wave includes the first frequency component whose frequency is the f and a second frequency component whose frequency is f′ where the f′ is a positive real number and is smaller than f,
the low frequency signal includes a first low frequency signal and a second low frequency signal,
the condition change unit decreases intensity of the first ultrasonic wave when the low frequency signal detector detects the first frequency signal having a frequency lower than f/r and having intensity not smaller than a predetermined threshold, and
the condition change unit stops emission of the first ultrasonic wave when the low frequency signal detector detects the second low frequency signal having a frequency lower than f′/r and having intensity not smaller than a predetermined threshold, or when the low frequency signal detector detects a predetermined change of the second low frequency signal.

13. The ultrasonic irradiation apparatus of claim 10, wherein the f=2f′.

14. The ultrasonic irradiation apparatus of claim 1, further comprising a notification unit which notifies a user that the condition change unit changes the ultrasonic setting value.

15. The ultrasonic irradiation apparatus of claim 1, further comprising an injection unit which injects, into the target portion, a micro substance which reflects or scatters the first ultrasonic wave.

16. A method for irradiating an ultrasonic wave by use of an ultrasonic irradiation apparatus comprising an ultrasonic emission part configured to emit an ultrasonic wave and an ultrasonic reception part configured to receive an ultrasonic wave, the method comprising:
setting an ultrasonic setting value including a frequency setting value for setting a first frequency f to be included in a first ultrasonic wave which is emitted from the ultrasonic emission part toward a target portion where the f is a real number;
causing the ultrasonic emission part to emit the first ultrasonic wave based on the ultrasonic setting value;
causing the ultrasonic reception part to receive a second ultrasonic wave travelling in a direction from the target portion;
extracting a low frequency signal included in the second ultrasonic wave, the low frequency signal having a frequency not greater than f/r where r is a real number and is greater than 2;
obtaining a peak value of the low frequency signal and a peak frequency corresponding to the peak value;
determining an occurrence status of a cavitation in an area between the ultrasonic emission part and the target portion, by comparing the peak value and the peak frequency with a predetermined threshold; and
changing the ultrasonic setting value based on a result of the determination.
